(19) United States
(12) Patent Application Publication
(10) Pub. No.: US 2012/0010750 A1
(43) Pub. Date: Jan. 12, 2012

(54) DEVICE AND METHOD FOR TESTING THE CLEANING EFFECT OF A CLEANING APPARATUS

(75) Inventors: Matthias Wahl, Langquaid (DE); Frank Winzinger, Regensburg (DE)

(73) Assignee: KRONES AG, Neutraubling (DE)

(21) Appl. No.: 13/150,509

(22) Filed: Jun. 1, 2011

(30) Foreign Application Priority Data
Jun. 1, 2010 (DE) 102010029576.0

(51) Int. Cl. G05B 23/02; G01N 33/00

(52) U.S. Cl. 700/266; 73/865.9

(57) ABSTRACT

The invention comprises a device for testing the cleaning effect of a cleaning apparatus, said cleaning apparatus being part of a container treatment plant, said device comprising a unit for introducing and/or applying a predetermined amount of contamination in and/or to a test container, and a unit for introducing the test container in the container treatment plant.

The present invention relates to a device and a method of testing the cleaning effect of a cleaning apparatus, said cleaning apparatus being part of a container treatment plant.

Cleaning apparatuses are used e.g. in filling plants of the beverage industry so as to clean and in particular sterilize containers, such as bottles. The cleaning effect of a cleaning apparatus is normally tested in that a predetermined amount of contamination is manually introduced in a test container by an operator. This test container is then, again manually, introduced in the cleaning apparatus and, after having been cleaned, it is examined in a laboratory with respect to the existing degree of residual contamination.

These known methods are, however, disadvantageous insofar as the handling of the test container through the operator may lead to an additional contamination of the test container, in particular a contamination through germs, whereby the test result may be corrupted and thus rendered inaccurate.

It is therefore the object of the present invention to provide a device and a method which allow the cleaning effect of a cleaning apparatus to be tested more effectively.

The invention provides a device used for testing the cleaning effect of a cleaning apparatus, said cleaning apparatus being part of a container treatment plant, and comprising a unit for introducing and/or applying a predetermined amount of contamination in and/or to a test container, and a unit for introducing the test container in the container treatment plant.

Due to the fact that the introduction and/or application of a predetermined amount of contamination and the introduction of the test container into the container treatment plant are carried out automatically, a contamination of the test container through an operator can be prevented or at least minimized and the cleaning effect of the cleaning apparatus can thus be tested more effectively.

The unit for introducing and/or applying a predetermined amount of contamination can therefore be implemented such that it places a predetermined number or mass of germs and/or a predetermined number or mass of dirt particles in and/or on the test container.

The predetermined amount of contamination may correspond to e.g. 10, 100, 1000, 10,000, 100,000, 1 million, 10 millions, 100 millions or 1 billion of germs and/or, depending on the size, to 10 to 1 million, in particular 100 to 1,000 dirt particles. Contamination may, in the widest sense of the word, also comprise fats, oils and other liquids or suspensions, of which amounts of 0.1 mg to 100 mg can be introduced by way of example.

The germs may correspond to one type of germs or to a mixture of several types of germs. The term germs may here stand for bacteria, spores of bacteria and/or viruses, especially pathogenic germs. In particular, germs of the type “Bacillus atrophaeus” and/or germs of the type “Bacillus subtilis” can be used.

The predetermined amount of contamination may e.g. be present in the form of a suspension, the unit for introducing and/or applying the predetermined amount of contamination introducing and/or applying the suspension in and/or to the test container.

Cleaning can here mean the removal of an amount of contamination in general. However, also sterilization may be referred to as cleaning. Also the removal of contaminations and sterilization may be referred to as cleaning.

The predetermined amount of contamination may comprise contaminations, such as dust and/or germs, e.g. germs of the type “Bacillus atrophaeus”.

The term cleaning effect may here stand for the cleaning accomplished, i.e. for example the percentage of the predetermined amount of dirt that has been removed by cleaning. In the case of sterilization also the sterility achieved can be referred to as cleaning effect, i.e. for example the percentage of the predetermined number of germs that has been removed and/or killed by cleaning.

The cleaning apparatus may comprise a plurality of cleaning elements. This allows a cleaning of containers at different positions in the container treatment plant.

The cleaning apparatus may comprise a rinser and/or a sterilization unit. The cleaning apparatus may also correspond to a rinser and/or a sterilization unit. In the case of a sterilization unit, the sterilization effect of the sterilization unit can be tested.

A sterilization unit may e.g. use hydrogen peroxide (H₂O₂) or peractetic acid or ozone or steam or foam as a sterilization agent. Alternatively or additionally, the sterilization unit may comprise one or a plurality of radiation sources for ultraviolet radiation and/or thermal radiation and/or microwave radiation and/or other kinds of radiation.

In the widest sense of the word, the cleaning apparatus may also comprise a heating device, e.g. a heating device of a blow moulding machine, or it may correspond to such a heating device, since heating of the containers in the heating device may result in an at least partial sterilization of the test container. Likewise, the cleaning apparatus may comprise one or a plurality of UV lamps and/or one or a plurality of microwave generators and/or a steam generator and/or a plasma generator and/or an electrode beam generator (E-beam) and/or an ozone generator. Also an X-ray and/or a gamma-ray generator would be imaginable in this context.

The test container may be a container which is adapted to be used during operation of the container treatment plant, in particular a preform of a blow moulding machine or a bottle. The method can thus be carried out with little effort.

The unit for introducing and/or applying a predetermined amount of contamination can be implemented such that the predetermined amount of contamination will be placed on a predetermined or desired surface area in and/or on the test container. The cleaning effect for specific surface areas of the container, e.g. for surface areas which are not easily accessible, can be tested in this way. The predetermined or desired surface area may comprise one subarea or a plurality of contiguous, partially contiguous or non-contiguous subareas.

Due to the automatic introduction and/or application of the predetermined amount of contamination, it may also be possible to achieve a more precise arrangement and/or a more precise dosage of the predetermined amount of contamination than by means of a manual arrangement executed by an operator.

The unit for introducing and/or applying a predetermined amount of contamination may especially comprise a plurality of elements for introducing and/or applying the predetermined amount of contamination. For example, different elements for introducing and/or applying the predetermined amount of contamination can be provided for different subareas in and/or on the test container. The predetermined amount of contamination can thus easily be placed in a plurality of subareas.

The unit for introducing and/or applying the predetermined amount of contamination may e.g. comprise a first element which applies a part of the predetermined amount of contamination to an outer surface of the test container and a second element which introduces a part of the predetermined amount of contamination in the test container, and which especially applies said part of the predetermined amount of contamination to an inner surface of the test container.

The unit for introducing and/or applying the predetermined amount of contamination and/or the unit for introducing the test container in the container treatment plant can be arranged and/or configured such that the introduction of the test container is carried out prior to, subsequent to and/or during the introduction and/or application of the predetermined amount of contamination.

For example, the unit for introducing and/or applying the predetermined amount of contamination may be arranged in the area of the conveying device of the container treatment plant and/or in the area of the unit for introducing the test container in the container treatment plant.

The device for testing the cleaning effect may, in addition, comprise a unit for discharging the test container from the container treatment plant. It is thus possible to discharge the test container from the container treatment plant at a desired point of the container treatment plant also during operation of the latter.

The unit for introducing the test container and/or the unit for discharging the test container may each comprise a plurality of elements which are used for introducing or discharging the test container and which can be arranged at different locations of the container treatment plant. In other words, the elements for introducing the test container and/or the elements for discharging the test container can be positioned variably. The test container can thus pass through different sections of the container treatment plant and this, in turn, allows the cleaning effect of cleaning elements to be tested in different sections of the container treatment plant.

The container treatment plant can correspond to a filling plant, in particular a filling plant of the beverage industry.

The container treatment plant may especially comprise a blow moulding machine for producing plastic containers from preforms, or it may correspond to such a blow moulding machine. The blow moulding machine may comprise a preform feeder, a heating device for heating the preforms and/or a blow module for blowing moulding, in particular for stretch blow moulding, the test containers in blow moulds.

The container treatment plant may additionally comprise a filler for filling containers, in particular plastic containers produced in the blow moulding machine. Furthermore, the container treatment plant may comprise an injection moulding machine for producing performs, a labeller, at least one conveying device and/or a regenerating device for regenerating returnable plastic bottles.

The device for testing the cleaning effect may also comprise a plurality of units for introducing and/or applying a predetermined amount of contamination. These units may especially be arranged at different positions, in particular within a container treatment plant.

One or a plurality of units for introducing and/or applying a predetermined amount of contamination may also be provided for operating and testing a plurality of respective container treatment plants.

A cleaning element of the container treatment plant may be arranged between a heating device of a blow moulding machine and a blow module of the blow moulding machine. Another cleaning element may e.g. be provided downstream of the blow module and upstream of a filler. Alternatively or additionally, a cleaning element can be arranged upstream of a regenerating device for regenerating returnable plastic bottles.

A unit for introducing the test container can be arranged and/or configured such that the test container is introduced upstream of, especially directly upstream of the cleaning apparatus, and in particular upstream of a predetermined cleaning element of the cleaning apparatus.

The unit for introducing the test container may especially be arranged and/or configured such that the test container is introduced upstream of, in particular directly upstream of a preform feeder, a heating device of a blow moulding machine, a labeller, a rinser, a sterilization unit, a container coating station, a filler and/or a capper or closure device. The unit for introducing the test container may also be arranged and/or configured such that the test container is introduced downstream of, in particular directly downstream of a filler, a rinser, a sterilization unit, a container coating station, a labeller, a blow module or in a preform feeder or between a heating device of a blow moulding machine and a blow module.

The unit for discharging the test container can be arranged and/or configured such that the test container is discharged downstream of, in particular directly downstream of the cleaning apparatus, in particular downstream of a predetermined cleaning element of the cleaning apparatus.

The unit for discharging the test container can especially be arranged and/or configured such that the test container is discharged upstream of, in particular directly upstream of a heating device of a blow moulding machine, a filler or a capper or closure device. The unit for discharging the test container may also be arranged and/or configured such that the test container is discharged downstream of, in particular directly downstream of a capper or closure device, a rinser, a sterilization unit, a container coating station, a labeller, a blow module and/or a filler or in a preform feeder or between a heating device of a blow moulding machine and a blow module.

When the container treatment plant comprises a packing device, the test container may be introduced upstream of, in particular directly upstream of the packing device, and discharged downstream of, in particular directly downstream of the packing device. If only closed containers are to be packed in the packing device so as to form packs, the predetermined amount of contamination may in this case also be applied only to the outer side of the test container, in particular to a closure of the test container and/or a label of the test container.

The container treatment plant may also comprise a blow moulding machine which includes an inmould labelling element. In the case of inmould labelling, labels are introduced in the blow moulds of the blow module. In this case, the application of the predetermined amount of contamination to the test container may comprise an application of the predetermined amount of contamination to a label before the label is introduced in a blow mould.

If the plant comprises an injection moulding machine for producing preforms, the test container can be introduced downstream of, in particular directly downstream of the injection moulding machine.

The device for testing the cleaning effect may additionally comprise a unit for determining an amount of residual contamination after cleaning of the test container by means of the cleaning apparatus.

The unit for determining an amount of residual contamination may comprise e.g. an optical sensor and/or a detector for biological and/or chemical contaminations, such as a sniffer. It is thus possible to avoid another direct interaction between the test container and an operator for determining the amount of residual contamination, whereby a further possible contamination of the test container can be avoided or at least reduced.

The unit for discharging the test container and/or the unit for determining an amount of residual contamination may be arranged and/or configured such that the determination of the amount of residual contamination is executed prior to, subsequent to and/or during the discharge of the test container from the container treatment plant. The unit for discharging the test container and/or the unit for determining an amount of residual contamination may additionally comprise a buffer for one or a plurality of test containers, in which said one or said plurality of test containers has introduced therein a nutrient solution so that, after a specified period of time, an increase in the number of germs can be observed or measured. The increase in the number of germs can especially also be observed or measured, in particular automatically, via the unit for determining the amount of residual contamination.

The device for testing the cleaning effect may additionally comprise a control element which is configured such that at least one operating parameter of the container treatment plant, in particular of the cleaning apparatus, is controlled, in particular by closed loop control, in dependence upon the determined amount of residual contamination. The efficiency of the cleaning machine can be improved in this way.

The control element can be configured such that it carries out a comparison of the determined amount of residual contamination with a predetermined value, in particular with the predetermined amount of contamination. The cleaning effect of the cleaning apparatus can be quantified in this way.

The control element can be configured such that, if the comparison of the determined amount of residual contamination with the predetermined value does not fulfil a predetermined criterion, it will output data to an operator, e.g. in the form of an optical and/or acoustic warning signal. It is thus possible to inform e.g. an operator, if the amount of residual contamination should become excessively high.

If it should e.g. be determined that the determined amount of residual contamination exceeds a predetermined threshold value, the cleaning effect of the cleaning apparatus can be increased by adapting at least one operating parameter of the cleaning apparatus and/or of the container treatment plant. If it should, however, be determined that the determined amount of residual contamination is smaller than a predetermined threshold value, the cleaning effect of the cleaning apparatus may also be reduced, whereby e.g. the amount of cleaning agents consumed in the cleaning apparatus can be reduced. To this end, the unit for determining an amount of residual contamination as well as the unit for introducing or discharging the test container or the unit for introducing and/or applying a predetermined amount of contamination may comprise an additional control element which is connected to the above-mentioned control element.

The at least one operating parameter may correspond to a concentration of a sterilization liquid, an amount of a sterilization liquid, a temperature of a sterilization liquid, an injection pressure of a sterilization liquid, a position on the test container for applying the predetermined amount of contamination, e.g. above an insertion depth of a lance, a radiation intensity of an UV lamp and/or of a microwave unit, a type of sterilization medium for the containers, e.g. hydrogen peroxide, peractetic acid or soap suds, a sterilization unit used, a cleaning time for the devices and/or the containers, a blowing pressure of a sterilization liquid and/or an ambient pressure in the container treatment plant or in a part of the container treatment plant. The ambient pressure in the container treatment plant or in a part of the container treatment plant may influence the sterility of the atmosphere in the container treatment plant or in a part of the container treatment plant. For example, an excess pressure in the container treatment plant or in a part of the container treatment plant may prevent or at least impede an ingress of contaminations.

The unit for introducing and/or applying the predetermined amount of contamination may comprise a drying element for drying the test container. Making use of this drying element it is possible to dry e.g. the test container when the predetermined amount of contamination has been introduced in and/or applied to the test container in the form of a suspension. The drying element may also correspond to the heating device of a blow moulding machine.

The unit for introducing and/or applying the predetermined amount of contamination may comprise one or a plurality of capillary tubes in a supply. This principle is known e.g. for a different case of use from DE 10 2008 037160 A1 and serves to accomplish very accurate dosing.

Alternatively or additionally, the unit for introducing and/or applying the predetermined amount of contamination may comprise a flow meter. By means of this flow meter, precise dosing of the predetermined amount of contamination can be achieved. In particular, the flow meter can be an inductively measuring flow meter.

Alternatively or additionally, the unit for introducing and/or applying the predetermined amount of contamination may comprise a weighing cell used in particular for determining the predetermined amount of contamination. It is thus possible to measure the weight of an amount of contamination introduced in the test container and to stop the introduction via a control, in particular a closed-loop control, when a predetermined threshold value has been reached.

Alternatively or additionally, the unit for introducing and/or applying the predetermined amount of contamination may comprise an optical sensor, such as a camera, the introduced amount of contamination being especially determined via the optical sensor, e.g. the camera, and the introduction being stopped via a control, in particular a closed-loop control, when a predetermined threshold value has been reached. To this end, the contamination can be dyed. The amount of contamination introduced can e.g. be determined in accordance with a method according to CN1654962 (A), in the case of which a CCD high-speed camera is used.

For determining the introduced amount of contamination, one or a plurality of piezoelectric metering valves can be used alternatively or additionally.

The unit for introducing and/or applying the predetermined amount of contamination may comprise at least one nozzle. The at least one nozzle can be arranged above a conveying device for conveying the test container.

Alternatively or additionally, the unit for introducing and/or applying the predetermined amount of contamination may comprise at least one contact element, the at least one contact element and/or the unit for introducing and/or applying the predetermined amount of contamination being configured such that the at least one contact element can be brought into direct contact with a surface of the test container and applies, through said direct contact, the predetermined amount of contamination, at least partially, to the surface. The at least one contact element may comprise especially a porous material, such as foamed plastic. The predetermined amount of contamination can thus, when provided in the form of a suspension, be taken up by the at least one contact element. The at least one contact element can be configured as a roller, which is adapted to be moved on a surface of the test container in rolling contact therewith.

The unit for introducing and/or applying the predetermined amount of contamination can be configured as a rotary-type unit or as a linear unit.

The unit for introducing and/or applying the predetermined amount of contamination and/or the unit for introducing the test container may each comprise one or a plurality of treatment elements of the container treatment plant or they may correspond to such treatment elements. By making use of a treatment element of the container treatment plant, cost of material can be saved.

A unit for introducing and/or applying the predetermined amount of contamination may e.g. correspond to a preform rinser or comprise a preform rinser. To this end, the preform rinser may, in particular temporarily, be configured such that it emits, e.g. instead of ionized air, the predetermined amount of contamination, e.g. in the form of a suspension.

The unit for introducing and/or applying the predetermined amount of contamination may be configured such that the test container is seized or held on the outer surface of its body, in particular such that predetermined or desired surfaces of the test container are exposed. In this way, it can be guaranteed that the predetermined amount of contamination will be placed, as far as possible, onto all critical surfaces of the test container, e.g. one the outer surface of the mouthpiece and/or on the complete inner surface.

The unit for introducing and/or applying the predetermined amount of contamination may also comprise a heating mandrel and/or a stretching rod of a blow moulding machine of the container treatment plant.

The unit for introducing the test container may e.g. comprise a preform feed or correspond to the latter.

The device for testing the cleaning effect may additionally comprise an element for cleaning, in particular sterilizing, the test container before the predetermined amount of contamination is introduced and/or applied. The cleaning effect of the cleaning apparatus can thus be tested even more effectively, since a possibly existing contamination of the test container is removed or at least minimized before the predetermined amount of contamination is introduced and/or applied.

The device for testing the cleaning effect may additionally comprise a test container, the test container being in particular a measurement dummy.

The measurement dummy is here an element having the form of a preform or of a container. A measurement dummy may also have the same dimensions as a preform or a container, and in particular a measurement dummy may also comprise the same material as a preform or a container.

A measurement dummy may also be adapted to be blown up. In other words, the measurement dummy may be mouldable into a plastic container in a blow mould of a blow moulding machine. Alternatively, the measurement dummy may be configured such that it is not mouldable into a plastic container in a blow mould of a blow moulding machine, i.e. in particular such that it will not undergo any change of shape or undergo only a minor change of shape.

In and/or on the measurement dummy there can be arranged at least one measuring element for determining at least one parameter value for operating a blow moulding machine, a container treatment plant, a preform feeder, a heating device of a blow moulding machine, a rinser, a sterilization unit, a container coating station, a filler, a capper or closure device and/or a labeller. The measurement dummy can be moved through the container treatment plant or through parts of the container treatment plant and, in the course of this process, the measuring element can determine the value of at least one parameter. It is thus possible to execute a measurement of a position- and/or time-dependent parameter.

The at least one measuring element can especially be configured such that it is able to measure a number of germs and/or a mass or a number of dirt particles. The at least one measuring element can additionally be configured such that it is able to measure a pressure value, a temperature value, a value of a force, a value of a moment, a position value, a period of time, an amount of gas or liquid, in particular a sterilization liquid, e.g. a volume value, a speed, a radiation intensity and/or a field strength. The measurement dummy may in particular comprise a plurality of measuring elements, each of said measuring elements being able to determine or measure one or a plurality of the above-mentioned values.

The measurement dummy may additionally comprise a transmitter element for transmitting the at least one parameter value determined by the measuring element to an evaluation element.

The measurement dummy may additionally comprise a storage element for storing the at least one parameter value determined by the measuring element.

The measurement dummy may additionally comprise an interface, in particular an USB interface, for connecting the storage element to an evaluation element and/or an external storage element.

The measurement dummy may additionally comprise an energy supply element for supplying the at least one measuring element, the transmitter element, the storage element and/or the interface with electric energy.

The measurement dummy may be moved, at least partially, through a conveying device by means of which the articles to be processed are moved during operation.

In addition, the measurement dummy may be implemented and/or configured such that it transmits the at least one parameter value determined by the measuring element to an evaluation element. In particular, the determined amount of residual contamination can be transmitted to an evaluation element.

The test container, in particular the measurement dummy, may additionally comprise at least one element for cleaning conveying elements of a conveying device of the container treatment plant. Dirt residues, which deposit on the conveying elements, can thus be removed or at least minimized, when the test container is moved at least partially through the container treatment plant by means of the conveying device.

The at least one element for cleaning conveying elements of the conveying device may be a brush, by way of example.

The device for testing the cleaning effect may also be implemented and/or configured such that a plurality of test containers is used for testing the cleaning effect of the cleaning apparatus. A plurality of test containers can in particular be provided with different predetermined amounts of contamination and/or types of contamination. Alternatively or additionally, the predetermined amount of contamination for different test containers may be placed in different subareas. It is thus possible to test the cleaning effect for different types and degrees of contamination.

An above-described device for testing the cleaning effect may especially be part of a container treatment plant. In other words, the present invention additionally provides a system comprising a container treatment machine and an above-described device for testing the cleaning effect of a cleaning apparatus, the cleaning apparatus being part of the container treatment plant. The container treatment plant and/or the device for testing the cleaning effect of the cleaning apparatus may comprise one or a plurality of the above-described features.

The present invention additionally provides a method for testing the cleaning effect of a cleaning apparatus, wherein the cleaning apparatus is part of a container treatment plant, said method comprising:
automatic introduction and/or application of a predetermined amount of contamination in and/or to a test container; and
automatic introduction of the test container in the container treatment plant.

Due to the fact that the introduction and/or the application of the predetermined amount of contamination as well as the introduction of the test container in the container treatment plant are executed automatically, a contamination of the test container through an operator can be prevented or at least minimized, and the cleaning effect of the cleaning apparatus can thus be tested more effectively.

The method can especially be carried out by means of a device described hereinbefore.

After having been introduced in the container treatment plant, the test container can be treated by one or by a plurality of treatment elements of the container treatment plant, and in particular it can be treated in the same way as containers which are to be treated during operation of the container treatment plant.

The test container can especially be treated by predetermined treatment elements. In particular, the test container need not be filled. The increase in the number of germs can thus be measured in the way described hereinbefore. In principle, it would, however, also be possible to measure the increase in the number of germs when the product has been filled in the test container.

The method may especially comprise cleaning of the test container by means of the cleaning apparatus and determining an amount of residual contamination when the test container has been cleaned by the cleaning apparatus.

The determination of the amount of residual contamination can especially be carried out automatically, e.g. by a sensor. Alternatively or additionally, the amount of residual contamination can be determined in a laboratory.

The introduction of the test container can be carried out prior to, subsequent to and/or during the introduction and/or application of the predetermined amount of contamination.

The method may additionally comprise cleaning, in particular sterilizing, the test container prior to the introduction and/or application of the predetermined amount of contamination. It is thus possible to allow an even more effective testing of the cleaning effect of the cleaning apparatus, since a possibly existing contamination of the test container is removed or at least minimized prior to the introduction and/or application of the predetermined amount of contamination.

The method may additionally comprise discharging the test container from the container treatment plant, in particular prior to, subsequent to and/or during the determination of the amount of residual contamination.

The method may additionally comprise comparing the determined amount of residual contamination with a predetermined value, in particular with the predetermined amount of contamination. It is thus possible to quantify the cleaning effect of the cleaning apparatus.

In particular, the determined amount of residual contamination can be compared with a predetermined value. It is thus possible to classify the cleaning effect of the cleaning apparatus.

The comparison can be executed in particular automatically, e.g. by a control element. If the comparison of the determined amount of residual contamination with the predetermined value does not fulfil a predetermined criterion, e.g. data may be outputted to an operator, e.g. in the form of an optical and/or acoustic warning signal. It is thus possible to inform e.g. an operator, if the amount of residual contamination should become excessively high.

The predetermined amount of contamination may be placed at one or at a plurality of predetermined locations of the test container. In particular, the amount of contamination may be placed e.g. on container parts which are not easy to clean. The predetermined amount of contamination may be placed e.g. on the bottom and/or on one or a plurality of threads or mouthpieces of the test container.

The test container may have one or a plurality of undercuts; in particular, the predetermined amount of contamination is, at least partially, applied and/or introduced in the area of the at least one undercut.

The method may further comprise controlling, in particular by closed loop control, at least one operating parameter of the container treatment plant, in particular of the cleaning apparatus, in dependence upon the determined amount of residual contamination, in particular in dependence upon the result of the comparison between the determined amount of residual contamination and the predetermined value. The cleaning effect of the cleaning apparatus can be controlled in this way by open loop or closed loop control. In particular, it is thus possible to optimize the operation and/or the material usage of the cleaning apparatus.

In particular, it is possible to adjust or control, in particular by closed loop control, an operating parameter of at least one cleaning element of the cleaning apparatus on the basis of the determined amount of residual contamination, in particular on the basis of the result of the comparison between the determined amount of residual contamination and the predetermined value.

For example, the amount, the concentration and/or the temperature of a sterilization liquid and/or the injection pressure or blowing pressure of the sterilization liquid can be adjusted or controlled by open loop or closed loop control. Alternatively or additionally, it is possible to adjust or control by open loop or closed loop control an amount of sterilization liquid, a position on the test container for applying the predetermined amount of contamination, e.g. via an insertion depth of a lance, a radiation intensity of an UV lamp and/or a microwave unit, a type of sterilization medium for the containers, e.g. hydrogen peroxide or peractetic acid or soap suds, a sterilization unit used, a cleaning time for the devices and/or the containers, a blowing pressure of a sterilization liquid and/or an ambient pressure in the container treatment plant or in a part of the container treatment plant.

The method may additionally comprise cleaning one or a plurality of elements of the plant through which the test container has passed. Sterility of the machines in a subsequent production process can be guaranteed in this way. In particular, it is also possible to clean elements of the plant whenever a test container provided with a predetermined amount of contamination has passed therethrough, and the cleaning can especially be carried out by a CIP (cleaning in place) method. For example, heating mandrels, a valve block, the clean room, conveying elements and/or filling valves can be cleaned.

The introduction and/or discharge can especially be executed such that no additional contamination will be introduced in and/or applied to the test container. It is thus possible to avoid an additional contamination of the test container.

The test container may be a preform or a container, e.g. a bottle, that can be used during operation of the container treatment plant. This allows the method to be executed with little expenditure.

Alternatively, the test container may correspond to the above-described measurement dummy.

The above-described method may especially be executed with more than one test container. In particular, two test containers differing from one another can be used. It is thus possible to test different parts of the container treatment plant and the cleaning elements arranged therein. For example, one test container may be configured as a bottle and another one as a preform.

It is especially possible to automatically provide the at least two test containers differing from each other with a predetermined amount of contamination in succession and to automatically introduce them in the container treatment plant in succession.

A plurality of test containers can especially be provided with different predetermined amounts of contamination and/or types of contamination. Alternatively or additionally, the predetermined amount of contamination can be arranged in different subareas for different test containers. It is thus possible to test the cleaning effect for different types and degrees of contamination.

The introduction and/or discharge of the one or of the plurality of test containers can be executed at the above-described locations of the container treatment plant. In particular, the at least one test container can be introduced upstream of, especially directly upstream of the cleaning apparatus, in particular a predetermined cleaning element of the cleaning apparatus and discharged downstream of, especially directly downstream of the cleaning apparatus, in particular a predetermined cleaning element of the cleaning apparatus. The cleaning effect of the cleaning apparatus can be determined in this way.

One or a plurality of steps of an above-described method may also be executed more than once, especially repeatedly. For example, at least one test container may be introduced in the container treatment plant at different locations and/or discharged from the container treatment plant at different locations. This allows the cleaning effect of cleaning elements of the cleaning apparatus to be examined in different sections of the container treatment plant.

In particular, the method may be executed, depending on the requirements to be fulfilled, at predetermined intervals, especially between two production processes. An interval may e.g. amount to half an hour or a full hour, four, eight, twelve or 24 hours, but it may also be as long as one week up to one month. Alternatively or additionally, the method may be executed at irregular time intervals, e.g. between the filling processes of two different products on a filling line.

In the case of this method, the test containers may especially be provided with additional identification characteristics, such as RFID chips and/or bar codes, for the purpose of tracking, e.g. for laboratory purposes. The identification will be particularly suitable if different amounts of contamination are applied to a plurality of test containers, also for the purpose of comparing a plurality of cleaning apparatuses or elements.

Further advantages and features of the present invention will be explained hereinbelow on the basis of exemplary figures, in which:

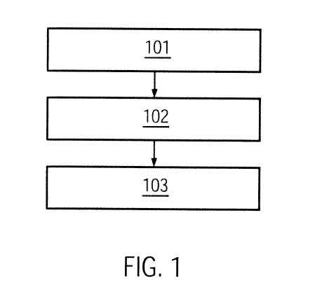

FIG. 1 shows, in the form of a flow diagram, an illustration of an exemplary method of testing the cleaning effect of a cleaning apparatus;

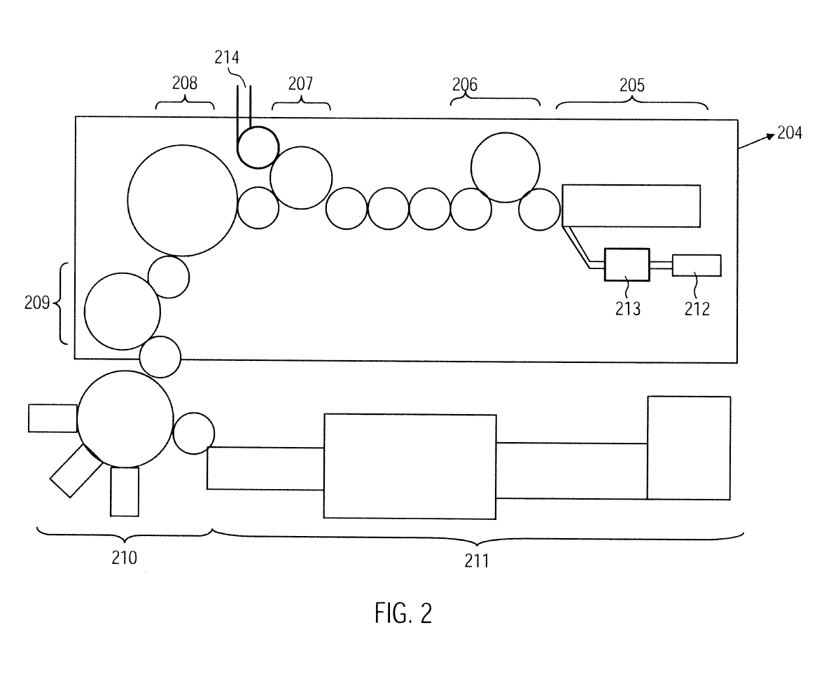

FIG. 2 shows an exemplary container treatment plant including a cleaning apparatus;

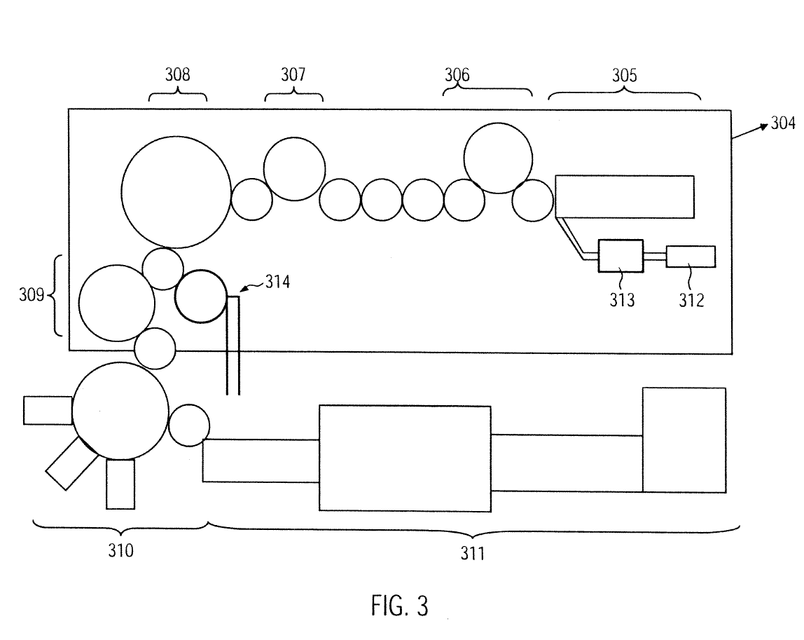

FIG. 3 shows a further exemplary container treatment plant including a cleaning apparatus;

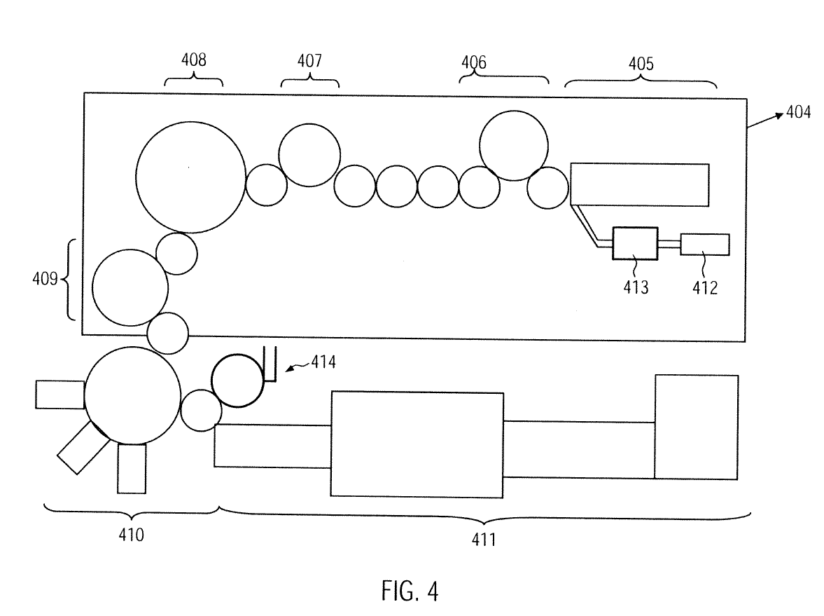

FIG. 4 shows a further exemplary container treatment plant including a cleaning apparatus;

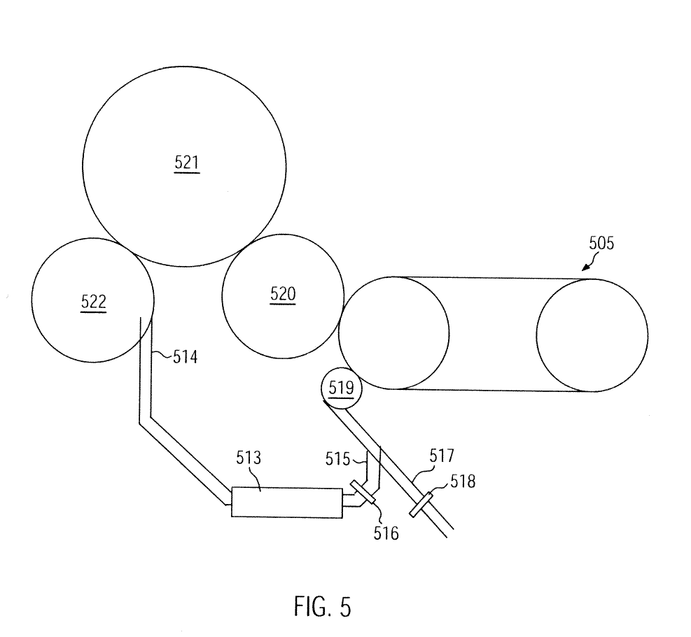

FIG. 5 shows an exemplary container treatment plant including a device for testing the cleaning effect of a cleaning apparatus;

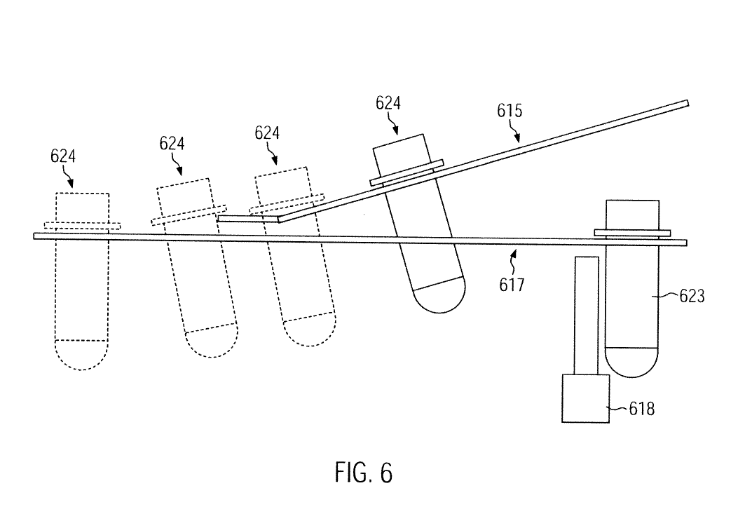

FIG. 6 shows an exemplary unit for introducing a test container;

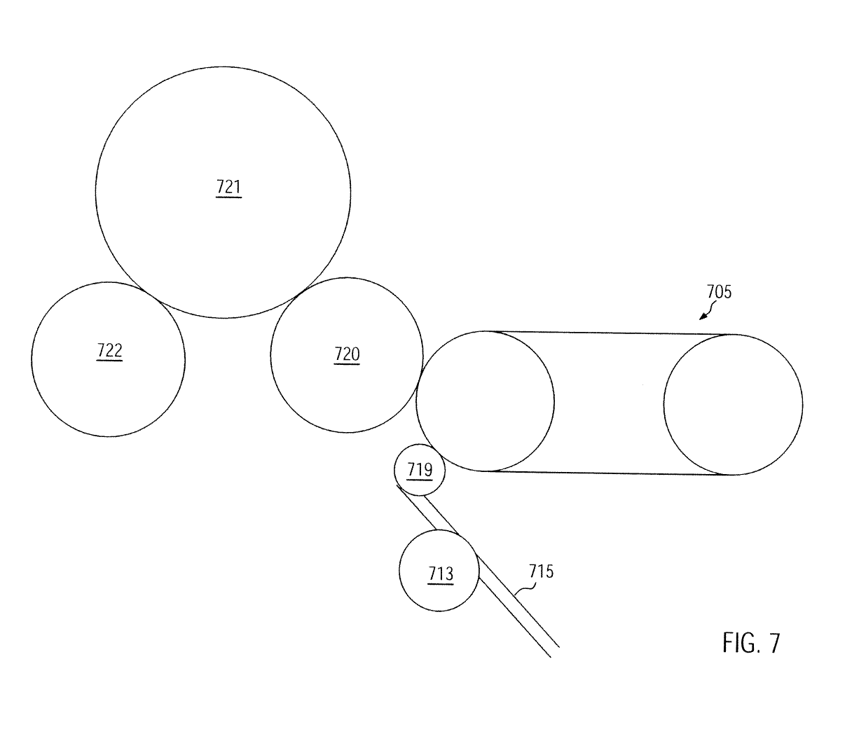

FIG. 7 shows a further exemplary container treatment plant including a device for testing the cleaning effect of a cleaning apparatus;

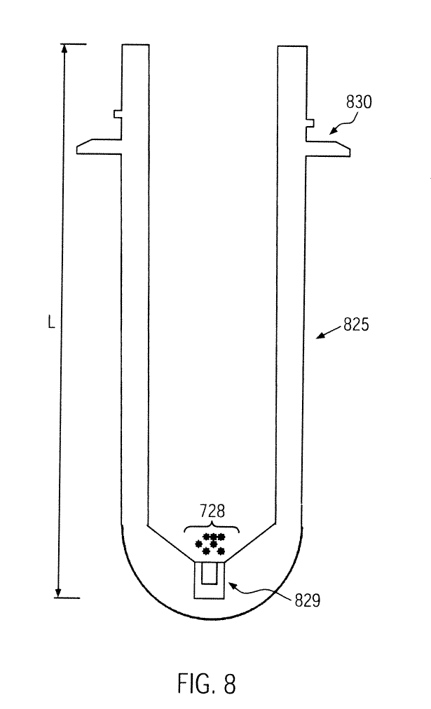

FIG. 8 shows an exemplary test container;

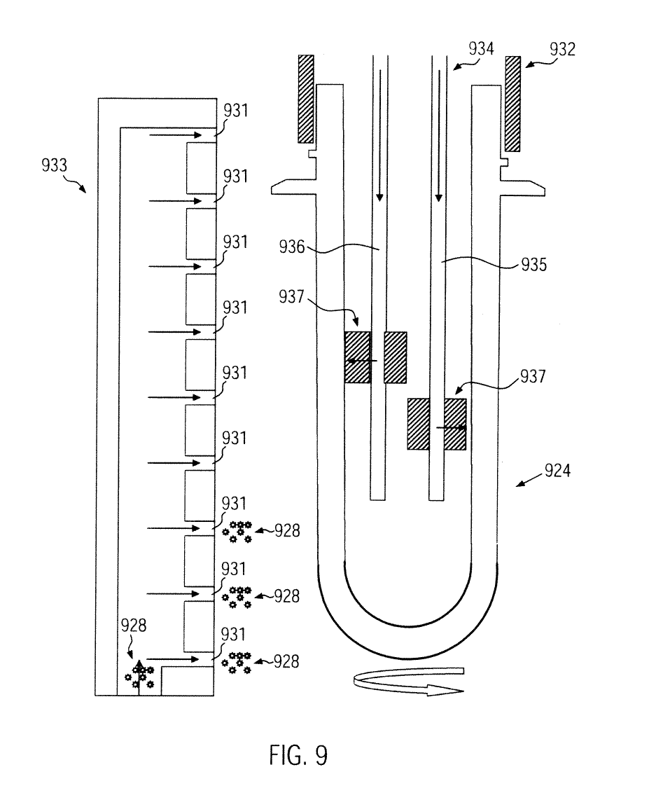

FIG. 9 shows an exemplary unit for introducing and/or applying a predetermined amount of contamination in and/or to a test container; and

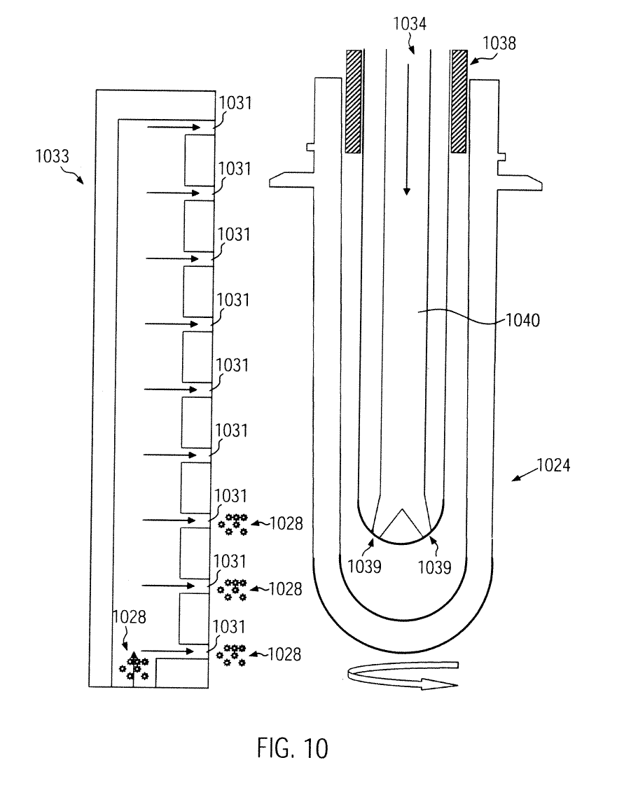

FIG. 10 shows a further exemplary unit for introducing and/or applying a predetermined amount of contamination in and/or to a test container.

FIG. 1 illustrates, in the form of a flow diagram, an exemplary method for testing the cleaning effect of a cleaning apparatus. The cleaning apparatus is part of a container treatment plant, e.g. a filling plant for beverages.

To begin with, a predetermined amount of contamination, e.g. a predetermined amount of germs and/or dirt particles, is automatically introduced in and/or applied to a test container in a first step 101.

The predetermined amount of contamination may correspond to e.g. 100.000 up to 1 million spores of bacteria in a suspension of from 10 to 100 microliter up to 10 milliliter liquid.

The test container may, for example, be a container for the cleaning of which the cleaning apparatus is provided. The test container may e.g. be a bottle, in particular a plastic bottle. Alternatively, the test container may also be a measurement dummy. The measurement dummy may comprise e.g. a measuring element for determining the amount of contamination, e.g. a number of germs or a number of dirt particles, and/or additional undercuts.

The test container is then automatically introduced in the container treatment plant in a step 102. Due to the fact that the introduction and/or the application of the predetermined amount of contamination as well as the introduction of the test container in the container treatment plant are executed automatically, a contamination of the test container through an operator can be prevented or at least minimized, and the cleaning effect of the cleaning apparatus can thus be tested more effectively.

In the present example, the test container is first intentionally provided with a defined amount of contamination and, subsequently, introduced in the container treatment plant. Alternatively, the step of introducing and/or applying a predetermined amount of contamination may also be executed during and/or after the automatic introduction of the test container into the container treatment plant.

In step 103, the amount of residual contamination in and/or on the test container is determined. The determination of the amount of residual contamination can be carried out prior to, subsequent to and/or during discharge of the test container from the container treatment plant.

At least one operating parameter of the container treatment plant, in particular of the cleaning apparatus, can be controlled in dependence upon the amount of residual contamination determined. The efficiency of the cleaning machine can be improved in this way.

If it should e.g. be determined that the determined amount of residual contamination exceeds a predetermined threshold value, the cleaning effect of the cleaning apparatus can be increased by adapting at least one operating parameter of the cleaning apparatus and/or of the container treatment plant. If it should, however, be determined that the determined amount of residual contamination is smaller than a predetermined threshold value, the cleaning effect of the cleaning apparatus may also be reduced, whereby e.g. the amount of cleaning agents consumed in the cleaning apparatus can be reduced.

The at least one operating parameter may be a concentration of a sterilization liquid, an amount of a sterilization liquid, a temperature of a sterilization liquid, an injection pressure of a sterilization liquid, a blowing pressure of a sterilization liquid and/or an ambient pressure in the container treatment plant or in a part of the container treatment plant.

FIGS. 2 to 4 show an exemplary container treatment plant, in particular a filling plant, comprising a preform heating device 205, 305, 405, a blow module 206, 306, 406, a cleaning element 207, 307, 407, a filler 208, 308, 408 and a capper or closure device 209, 309, 409, which are arranged in a clean room 204, 304, 404. The container treatment plant additionally comprises a labeller 210, 310, 410 and a packing device 211, 311, 411. Plastic preforms, from which plastic containers are formed in the blow module 206, 306, 406 by means of blowing, in particular stretch blow moulding, are introduced in the heating device 205, 305, 405 via a preform feeder 212, 312, 412.

FIGS. 2 to 4 additionally show a device for testing the cleaning effect of a cleaning apparatus of the container treatment plant, in particular of the cleaning element 207, 307, 407.

In the area of the preform feeder 212, 312, 412, a unit 213, 413, 513 is arranged, which is used for introducing and/or applying a predetermined amount of contamination in and/or to a test container. The unit 213, 413, 513 can especially be used for providing one or a plurality of preforms with a predetermined amount of contamination.

In the present example, preforms are used as test containers. Alternatively, also at least one measurement dummy may be used as a test container. In addition, the unit for introducing the test container corresponds to the preform feeder 212, 312, 412 in this example. The unit for introducing the test container may, however, also be provided as a separate element.

In addition to the cleaning element 207, 307, 407 also other cleaning elements may be provided in the container treatment plant.

FIGS. 2 to 4 additionally show units 214, 314, 414 for discharging the test container from the container treatment plant.

In FIG. 2, the unit 214 for discharging the test container is arranged immediately after the cleaning element 207. In FIG. 3, the unit 314 for discharging the test container is alternatively arranged after the filler 308. In FIG. 4, the unit 414 for discharging the test container is provided after the labeller.

The different points of arrangement of the units 214, 314, 414 for discharging the test container allow testing of the cleaning effect of different or of a plurality of cleaning elements of the container treatment plant, e.g. cleaning elements of the blow module 206, 306, 406, of the filler 208, 308, 408 or of the labeller 210, 310, 410. These additional cleaning elements are not shown in FIGS. 2 to 4.

The device for testing the cleaning effect may additionally comprise sensors by means of which an amount of residual contamination in and/or on the test container can be determined. A further direct interaction between the test container and an operator for determining the amount of residual contamination can thus be avoided, whereby a further possible contamination of the test container can be prevented or at least reduced.

FIG. 5 shows an exemplary container treatment plant in the form of a blow moulding machine and a device for testing the cleaning effect of a cleaning unit of the blow moulding machine, e.g. of the heating device or of a preform rinser, which is not shown.

The containers can be introduced in the exemplary blow moulding machine via an infeed wheel 519. On the one hand, preforms for producing plastic containers can be introduced in the blow moulding machine via a unit 517. On the other hand, test containers can be provided with a predetermined amount of contamination in a unit 513 for introducing and/or applying a predetermined amount of contamination in and/or to a test container, and introduced in the blow moulding machine via a unit 515 for introducing the test container. By means of stoppers 516 and 518, respectively, the transport paths for supplying the preforms or the test containers can be closed, in particular temporarily.

The blow moulding machine additionally comprises transfer elements 520 and 552.

After having passed through the heating device 505 and the blow moulding wheel 521, the test container can be discharged from the plant via a unit 514 for discharging the test container.

The test container may e.g. be a measurement dummy comprising a measurement element for determining an amount of residual contamination. The amount of residual contamination determined can be read out by connecting a terminal of the measurement dummy with contacts in the return path to the unit 513 for introducing and/or applying a predetermined amount of contamination in and/or to a test container, or it can be transmitted via a transmitter element of the measurement dummy, e.g. an RFID chip (radio frequency identity chip), an infrared transmitter or a bluetooth transmitter to an evaluation element, e.g. a personal computer (PC) or a memory programmable controller (PLC).

FIG. 6 shows an exemplary unit 615 for introducing the test container in a container treatment plant. In particular, a plurality of test containers 624 can be introduced in the container treatment plant in sequence. To this end, a stopper 618 can be provided, which interrupts the flow of preforms 623 that can be introduced in the container treatment plant via a unit 617.

The test containers 624 may e.g. be contaminated with different predetermined amounts of contamination and/or they may be provided in different subareas with a predetermined amount of contamination. The cleaning effect for different types and degrees of contamination can be tested in this way.

FIG. 7 shows a further exemplary container treatment plant in the form of a blow moulding machine. The blow moulding machine corresponds essentially to the blow moulding machine according to FIG. 5 and comprises an infeed wheel 719, a heating device 705, a blow moulding wheel 721 and transfer elements 720 and 722. The unit 713 for introducing and/or applying a predetermined amount of contamination in and/or to a test container is implemented as a rotary-type unit in this example.

The unit 713 may simultaneously also be a preform rinser, which is able to rinse preforms with ionized air or with a cleaning medium. The unit 713 can be implemented and/or configured such that, after the introduction and/or application of a predetermined amount of contamination in and/or to a test container, self-cleaning (cleaning in place, CIP) of the unit 713 is executed. A contamination of preforms during subsequent cleaning of the preforms can be prevented or at least minimized in this way.

An exemplary test container in the form of a measurement dummy 825 is shown in FIG. 8. The exemplary measurement dummy 825 has introduced therein a predetermined amount of germs 828, said germs 828 being preferably placed on the base of the measurement dummy 825. In addition, the exemplary measurement dummy 825 comprises a measurement element 829 for determining a number of germs.

The exemplary measurement dummy 825 additionally comprises a mouthpiece 830. In the area of the mouthpiece 830, the full predetermined amount of contamination or part of said amount of contamination can be placed. The mouthpiece 830 especially comprises a carrier ring, a support ring and a thread.

The shape and the geometrical dimensions of the exemplary measurement dummy 825 can correspond to those of a preform. Alternatively, the length of the measurement dummy 825 can correspond to the length of a plastic container to be produced.

In the area of the mouthpiece 830, the measurement dummy 825 may also comprise at least one brush. It is thus possible to clean conveying elements of the container treatment plant which come into contact with said brushes.

FIG. 9 shows an exemplary unit for introducing and/or applying a predetermined amount of contamination in and/or to a test container 924, said unit comprising a first element 933 and a second element 934. The test container 924 is here held by an outer gripper 932 and can thus be rotated, as is schematically indicated in the figure by an arrow below the test container 924.

The first element 933 of the exemplary unit for introducing and/or applying a predetermined amount of contamination is used for applying a contamination to the test container 924, especially to a surface of the outer face of said test container 924. In particular, a predetermined amount of contamination is here applied to the area of the mouthpiece from outside, since this area has to satisfy higher demands as regards cleaning.

The second element 934 of the exemplary unit for introducing and/or applying a predetermined amount of contamination is used for introducing a contamination into the test container 924, in particular to a surface of the inner face of the test container 924.

The first element 933 comprises a plurality of nozzles 931 through which a contamination can be applied to the test container 924 along the longitudinal axis of the test container 924. For example, germs 928 contained in a suspension can be sprayed onto the test container 924 through the nozzles 931 by means of compressed air.

The second element 934 is implemented in the form of a lance which moves into the test container 924. The lance comprises two channels 935 and 936 having each contact elements 937 arranged thereon. The contact elements 937 can be configured such that they are capable of taking up, at least partially, an amount of contamination introduced in the channels 935 and 936 and placing this amount of contamination onto a surface of the test container 924 by moving across said surface in rolling or wiping contact therewith. To this end, the contact elements 937 may comprise a porous or spongelike material. Through a predetermined contact pressure between the porous material and the inner face of the test container, a predetermined amount of contamination can be applied.

The amount of contamination can be fed into the channels and their outlets either at intervals, e.g. a short time before the contamination is applied, or with a constant volume flow.

The first element 933 may also comprise one or more contact elements instead of or in addition to the nozzles 931.

FIG. 10 shows a further exemplary unit for introducing and/or applying a predetermined amount of contamination in and/or to a test container 1024, said unit comprising a first element 1033 and a second element 1034. The test container 1024 is here held by a retaining mandrel 1038 and it can also be rotated by means of said retaining mandrel 1038, as is schematically indicated in said figure by an arrow below the test container. Alternatively, also the first element 1033 and/or the second element 1034 can be rotatable. Also a stationary insertion without rotation of one of the elements would be imaginable.

The first element 1033 corresponds essentially to the first element 933 in FIG. 9. In particular, the first element 1033 comprises a plurality of nozzles 1031.

The second element 1034 of the exemplary unit for introducing and/or applying a predetermined amount of contamination serves to introduce a contamination in a test container 1024, in particular onto a surface of the inner face of the test container 1024.

The second element 1034 is configured in the form of a lance which moves into the test container 1024. The lance comprises a plurality of nozzles 1039 by means of which a contamination can be introduced in the test container. To this end, the second element 1034 additionally comprises a channel 1040 through which the contamination is conducted to the nozzles 1039. The contamination can especially be provided in the form of a suspension.

It goes without saying that the features specified in the above-mentioned embodiments are not limited to these special combinations and can also be provided in arbitrary other combinations.

1. A device for testing the cleaning effect of a cleaning apparatus, said cleaning apparatus being part of a container treatment plant, comprising:
a unit (213; 313; 413; 513; 713) for introducing and/or applying a predetermined amount of contamination in and/or to a test container (624; 825; 924; 1024); and
a unit (515; 715) for introducing the test container (624; 825; 924; 1024) in the container treatment plant.

2. A device according to claim 1, further comprising a unit for determining an amount of residual contamination when the test container (624; 825; 924; 1024) has been cleaned by the cleaning apparatus.

3. A device according to claim 1 or 2, further comprising a unit (214; 314; 414; 514) for discharging the test container (624; 825; 924; 1024) from the container treatment plant.

4. A device according to one of the claim 2 or 3, further comprising a control element which is configured such that at least one operating parameter of the container treatment plant, in particular of the cleaning apparatus, is controlled, in particular by closed loop control, in dependence upon the determined amount of residual contamination.

5. A device according to claim 4, wherein the at least one operating parameter corresponds to a concentration of a sterilization liquid, an amount of a sterilization liquid, a temperature of a sterilization liquid, an injection pressure of a sterilization liquid, a blowing pressure of a sterilization liquid and/or an ambient pressure in the container treatment plant.

6. A device according to one of the preceding claims, wherein the unit (213; 313; 413; 513; 713) for introducing and/or applying a predetermined amount of contamination comprises a drying element for drying the test container (624; 825; 924; 1024).

7. A device according to one of the preceding claims, wherein the unit (213; 313; 413; 513; 713) for introducing and/or applying a predetermined amount of contamination comprises a treatment element of the container treatment plant.

8. A device according to one of the preceding claims, further comprising the test container (624; 825; 924; 1024), said test container (624; 825; 924; 1024) being in particular a measurement dummy.

9. A system comprising a container treatment plant and a device according to one of the preceding claims.

10. A method of testing the cleaning effect of a cleaning apparatus, wherein the cleaning apparatus is part of a container treatment plant, said method comprising:
automatic introduction and/or application of a predetermined amount of contamination in and/or to a test container (624; 825; 924; 1024); and
automatic introduction of the test container (624; 825; 924; 1024) in the container treatment plant.

11. A method according to claim 10, further comprising determining an amount of residual contamination when the test container (624; 825; 924; 1024) has been cleaned by means of the cleaning apparatus.

12. A method according to claim 10 or 11, wherein the test container (624; 825; 924; 1024) is introduced prior to, subsequent to and/or during the introduction and/or application of the predetermined amount of contamination.

13. A method according to one of the claims 10-12, further comprising discharging the test container (624; 825; 924; 1024) from the container treatment plant, in particular prior to, subsequent to and/or during the determination of the amount of residual contamination.

14. A method according to one of the claims 11-13, further comprising comparing the determined amount of residual contamination with a predetermined value, in particular with the predetermined amount of contamination.

15. A method according to one of the claims 10-14, further comprising controlling, in particular by closed loop control, at least one operating parameter of the container treatment plant, in particular of the cleaning apparatus, in dependence upon the determined amount of residual contamination, in particular in dependence upon the result of the comparison between the determined amount of residual contamination and the predetermined value.

16. A method according to one of the claims 10-15, further comprising cleaning one or a plurality of elements of the plant through which the test container (624; 825; 924; 1024) has passed.
